(19) United States
(12) Patent Application Publication
(10) Pub. No.: US 2023/0351885 A1
(43) Pub. Date: Nov. 2, 2023

(54) SENSING DATA MANAGEMENT SYSTEM, SENSING DATA TRANSMISSION METHOD, SENSING DEVICE, INFORMATION COMMUNICATION TERMINAL, AND PROGRAM

(71) Applicant: OMRON HEALTHCARE Co., Ltd., Kyoto (JP)

(72) Inventor: Tetsuzo OKAZAKI, Kyoto (JP)

(21) Appl. No.: 18/346,607

(22) Filed: Jul. 3, 2023

Related U.S. Application Data
(63) Continuation of application No. PCT/JP2021/046783, filed on Dec. 17, 2021.

(30) Foreign Application Priority Data
Jan. 8, 2021 (JP) 2021-001980

(51) Int. Cl. G08C 19/00; G06F 3/14; A61B 5/00

(52) U.S. Cl. CPC G08C 19/00; G06F 3/14; A61B 5/0022; G06K 7/1417

(57) ABSTRACT

Sensing data can be transmitted in association with unique identification information of a subject without causing an increase in costs for a sensing device and burden of costs incurred to the subject. An information communication terminal reads an information code obtained by encoding at least address information of the sensing device and transmits notification information including at least unique identification information of the subject to the sensing device based on the address information represented by the information code. Upon receiving the notification information transmitted from the information communication terminal, the sensing device transmits the sensing data of the subject to a data processing device together with the unique identification information included in the notification information.

### CROSS-REFERENCE TO RELATED APPLICATIONS

This application is the U.S. national stage application filed pursuant to 35 U.S.C. 365(c) and 120 as a continuation of International Patent Application No. PCT/JP2021/046783, filed Dec. 17, 2021, which application claims priority to Japanese Patent Application No. 2021-001980, filed Jan. 8, 2021, which applications are incorporated herein by reference in their entireties.

### TECHNICAL FIELD

The present invention relates to a sensing data management system in which a data processing device collects and manages sensing data acquired by a sensing device, and a sensing data transmission method, a sensing device, an information communication terminal, and a program that are used in the sensing data management system.

### BACKGROUND ART

In recent years, a system in which a sensing device such as a blood pressure monitor installed in a hospital, a clinic, or the like is provided with a communication module for communicating with a mobile communication network, the communication module transmits sensing data such as measured blood pressure data of a subject to a server device via the network, and the server device manages various health conditions of the subject based on the blood pressure data has been proposed.

Typical blood pressure monitors do not have a section that receives input of unique identification information of subjects. For this reason, the server device is not able to associate blood pressure data received from the blood pressure monitor with the subjects.

Thus, for example, a method in which measured blood pressure data is temporarily imported to a mobile terminal such as a smartphone of the subject, and the blood pressure data is associated with identification information unique to the subject registered in the terminal in advance on the mobile terminal and then transmitted to a server device has been proposed in the related art (see Patent Document 1: JP 2018-108123 A).

### SUMMARY OF INVENTION

### Technical Problem

However, in the method described in Patent Document 1, not only is the communication module not being used even though the blood pressure monitor includes the communication module, but also the blood pressure monitor is to be provided with an additional wireless module, for example, a near-field wireless communication module in order to transfer the blood pressure data measured by the blood pressure monitor to a mobile terminal. For this reason, costs for the blood pressure monitor increase.

The present invention has been made in view of the above problems, and an object thereof is to provide a technique for associating sensing data with identification information unique to a subject of the sensing data without incurring an increase in costs for a sensing device.

### Solution to Problem

In order to solve the above problems, a first aspect of the present invention relates to a sensing data management system including a sensing device that measures sensing data of a subject, a data processing device that can perform data transmission with the sensing device via a network, and an information communication terminal that is used by the subject and can perform communication with the sensing device via the network.

The system includes a code holder that holds, in a readable state, an information code obtained by encoding at least address information of the sensing device.

The information communication terminal includes a storage that stores unique identification information of the subject, a reading processing unit for codes, and a notification information transmission processing unit. The reading processing unit for codes reads the information code from the code holder. The notification information transmission processing unit generates notification information including at least the unique identification information of the subject stored in the storage and transmits the notification information to the sensing device based on the address information included in the information code, in response to a reading operation of the information code.

The sensing device includes a notification information reception processing unit and a sensing data transmission processing unit. The notification information reception processing unit receives and stores the notification information transmitted from the information communication terminal via the network. The sensing data transmission processing unit transmits the sensing data of the subject to the data processing device together with the unique identification information included in the notification information.

The data processing device includes a sensing data reception processing unit. The sensing data reception processing unit receives the sensing data and the unique identification information transmitted from the sensing device via the network and holds the sensing data in association with the unique identification information.

According to the first aspect of the present invention, for example, when an administrator of the sensing device performs an operation of reading the information code by using the information communication terminal having a code reading function like a smartphone, notification information including the unique identification information of the subject stored in advance in the information communication terminal is transmitted to the sensing device based on the address information of the sensing device included in the information code. Then, the sensing device transmits the sensing data of the subject to the data processing device together with the unique identification information of the subject included in the notification information.

Thus, the sensing device does not need to transfer the sensing data to the information communication terminal and thus does not need to be provided with an additional communication module, and thereby product costs of the sensing device can be kept low.

According to a second aspect of the present invention, the code holder may be a printed medium on which the information code is displayed in a state of being optically readable by the information communication terminal.

According to a third aspect of the present invention, the code holder may be a wireless tag that outputs the information code as a wireless signal readable by the information communication terminal.

According to the second and third aspects of the present invention, the code holder can be manufactured separately from the sensing device, arranged at any position, and can also be added to an existing sensing device at any time.

A fourth aspect of the present invention relates to an information communication terminal used in a system including a sensing device that measures sensing data of a subject, a data processing device that can perform information communication with the sensing device via a network, the information communication terminal that can perform information communication with the data processing device via the network, and a code holder that holds, in a readable state, an information code obtained by encoding at least address information of the sensing device and includes a storage that stores unique identification information of the subject, a reading processing unit for codes, and a notification information transmission processing unit. The reading processing unit reads the information code from the code holder. The notification information transmission processing unit generates notification information including at least the unique identification information of the subject stored in the storage and transmits the notification information to the sensing device based on the address information included in the information code, in response to a reading operation of the information code.

According to the fourth aspect of the present invention, the unique identification information of the subject is automatically transmitted from the information communication terminal to the sensing device just by the subject performing an operation of reading the information code using his/her own information communication terminal. Thus, the subject does not need to manually input his/her own unique identification information to the sensing device, for example, thereby suppressing erroneous input of the unique identification information and reducing the operation burden on the subject.

A fifth aspect of the present invention further includes a position information measurement processing unit, and the position information measurement processing unit measures position information corresponding to the installation location of the sensing device in conjunction with reading processing of the information code. Then, the notification information transmission processing unit generates the notification information including the unique identification information of the subject stored in the storage and the position information and transmits the notification information to the sensing device based on the address information included in the information code.

According to the fifth aspect of the present invention, the sensing device can transmit the position information together with the unique identification information included in the notification information when transmitting the sensing data of the subject to the data processing device. Accordingly, the data processing device can, for example, estimate the name or address of the facility representing the sensing location of the subject from the map information based on the position information received together with the sensing data.

A sixth aspect of the present invention relates to a sensing device used in a system including the sensing device that measures sensing data of a subject, a data processing device that can perform data transmission with the sensing device via a network, and an information communication terminal that is used by the subject and can perform communication with the sensing device via the network.

The sensing device includes an information code output processing unit, a notification information reception processing unit, and a sensing data transmission processing unit. The information code output processing unit outputs an information code obtained by encoding at least address information of the sensing device in a state of being readable by the information communication terminal in association with a measurement operation of the sensing data. The notification information reception processing unit receives and stores notification information transmitted from the information communication terminal based on the information code via the network. The notification information includes at least unique identification information of the subject. The sensing data transmission processing unit transmits the sensing data of the subject to the data processing device together with the unique identification information of the subject included in the notification information.

According to the sixth aspect of the present invention, because the information code output processing unit that outputs the information code to the sensing device is provided, the sensing device can control a timing at which the information code is output.

In addition, the information code is output in association with a measurement operation of the sensing data, and the unique identification information of the subject is acquired from the information communication terminal in response to the information code. Thus, each time measurement of sensing data is performed, the unique identification information of the corresponding subject can be acquired, and thereby the unique identification information of the subject can be accurately associated with each piece of sensing data.

According to a seventh aspect of the present invention, the information code output processing unit may cause a display unit of the sensing device to display the information code in a state of being optically readable by the information communication terminal.

According to the seventh aspect of the present invention, because the sensing device has the information code displayed on its display, the subject can easily recognize the information code and can simply read the information code in a familiar operation by using the existing optical code reading function of the information communication terminal.

According to an eighth aspect of the present invention, the information code output processing unit may end an output of the information code when a first time set in advance elapses after an output of the information code is started or when the notification information transmitted from the information communication terminal is received.

According to the eighth aspect of the present invention, if the notification information is not received within the first time after the information code is displayed, the display of the information code is deleted. Thus, for example, even if a sensing operation is performed with no notification information including the unique identification information of the subject received from the information communication terminal, it is possible to suppress a trouble that the sensing data is transmitted to the data processing device without identification.

According to a ninth aspect of the present invention, if measurement of the sensing data is not performed within a second time set in advance from a time point at which the notification information is stored, the notification information reception processing unit may delete the stored notification information.

According to the ninth aspect of the present invention, if a sensing operation is not performed within the second time after the notification information is received, the stored notification information is deleted. Thus, it is possible to suppress a trouble that unique identification information of the subject unnecessarily remains in the sensing device and thus the unique identification information is transmitted in association with sensing data of another subject.

According to a tenth aspect of the present invention, if re-measurement of the sensing data is not performed within a third time set in advance after the transmission of the sensing data is ended, the notification information reception processing unit may delete the stored notification information.

According to the tenth aspect of the present invention, if one subject performs measurement of sensing data again within the third time, the measurement of sensing data can be performed without performing an operation of reading information code again, and thereby an operation burden on the subject can be reduced.

An eleventh aspect of the present invention relates to a sensing data management system including a sensing device that measures sensing data of a subject, a data processing device that can perform data transmission with the sensing device via a network, and an information communication terminal that is used by the subject and can perform communication with the sensing device via the network. The sensing data management system includes a code holder that holds, in a readable state, an information code obtained by encoding at least individual identification information of the sensing device and address information of the data processing device.

The information communication terminal includes a storage that stores unique identification information of the subject. In addition, when the information communication terminal reads the information code from the code holder, the information communication terminal generates first notification information including at least the individual identification information of the sensing device included in the information code and the unique identification information of the subject stored in the storage and transmits the first notification information to the data processing device based on the address information included in the information code, in response to a reading operation of the information code.

Meanwhile, when the data processing device receives the first notification information transmitted from the information communication terminal via the network, the data processing device acquires the address information of the sensing device associated in advance with the individual identification information of the sensing device included in the first notification information, generates second notification information including at least the unique identification information of the subject included in the first notification information, and transmits the second notification information to the sensing device based on the address information of the sensing device.

The sensing device receives the second notification information transmitted from the data processing device and transmits the sensing data of the subject to the data processing device together with the unique identification information of the subject included in the second notification information.

According to the eleventh aspect of the present invention, because the address information of the sensing device, for example, the telephone number and the email address are collectively managed by the data processing device, there is no concern that the address information of the sensing device may leak from the information code, and thereby unauthorized access by others to the sensing device can be suppressed.

An twelfth aspect of the present invention relates to a sensing data management system including a sensing device that measures sensing data of a subject, a data processing device that can perform data transmission with the sensing device via a network, and an information communication terminal that is used by the subject and can perform communication with the sensing device via the network. The sensing data management system includes a code holder that holds, in a readable state, an information code obtained by encoding at least individual identification information of the sensing device and address information of the data processing device.

The information communication terminal includes a storage that stores unique identification information of the subject. In addition, when the information communication terminal reads the information code from the code holder, the information communication terminal generates first notification information including at least the individual identification information of the sensing device included in the information code and the unique identification information of the subject stored in the storage and transmits the first notification information to the data processing device based on the address information included in the information code.

When the data processing device receives the first notification information transmitted from the information communication terminal, the data processing device acquires the address information of the sensing device associated in advance with the individual identification information of the sensing device included in the first notification information and transmits second notification information including at least the unique identification information of the subject included in the first notification information to the sensing device based on the address information of the sensing device.

The sensing device receives the second notification information transmitted from the data processing device via the network and transmits the sensing data of the subject to the data processing device together with the unique identification information of the subject included in the second notification information.

According to the twelfth aspect of the present invention, the sensing device does not need to perform processing of receiving the notification information and processing of associating the sensing data of the subject with the unique identification information, and thus the processing burden on the sensing device can be reduced. In addition, there is no need to assign address information to the sensing device, and thereby unauthorized access by others to the sensing device can be suppressed.

A thirteenth aspect of the present invention relates to a sensing data management system including a sensing device that measures sensing data of a subject, a data processing device that can perform data transmission with the sensing device via a network, and an information communication terminal that is used by the subject and can perform communication with the sensing device via the network.

The sensing device generates an information code obtained by encoding at least the sensing data and address information of the data processing device and outputs the information code in a state of being readable by the information communication terminal.

The information communication terminal includes a storage that stores unique identification information of the subject. In addition, when the information communication terminal reads the information code, the information communication terminal generates notification information in which the sensing data included in the information code is associated with the unique identification information of the subject stored in the storage and transmits the notification information to the data processing device based on the address information included in the information code.

The data processing device receives the notification information transmitted from the information communication terminal via the network and stores the sensing data included in the notification information and the unique identification information of the subject in a state of being associated with each other.

According to the thirteenth aspect of the present invention, the information communication terminal performs the processing of associating the sensing data of the subject measured by the sensing device with the unique identification information of the subject. In addition, the sensing device is provided with a function of generating and displaying an information code including the sensing data, and there is no need to additionally provide, in the sensing device, a wireless module such as Bluetooth, in order to transfer the sensing data to the information communication terminal. Thus, it is possible to eliminate the need for any communication module from the sensing device, and thereby lower costs can be incurred for the sensing device.

### Advantageous Effects of Invention

In other words, according to this invention, sensing data can be associated with identification information unique to the subject of the data without incurring an increase in costs for a sensing device.

### BRIEF DESCRIPTION OF DRAWINGS

Various embodiments are disclosed, by way of example only, with reference to the accompanying schematic drawings in which corresponding reference symbol s indicate corresponding parts, in which:

FIG. 1 is a diagram illustrating an overall configuration of a sensing data management system according to a first embodiment of the invention;

FIG. 2 is a block diagram illustrating a hardware configuration of a sensing device used in the system illustrated in FIG. 1;

FIG. 3 is a block diagram illustrating a software configuration of the sensing device used in the system illustrated in FIG. 1;

FIG. 4 is a diagram illustrating an example of a QR code (trade name) used as an information code;

FIG. 5 is a diagram illustrating an example of a bar code used as an information code;

FIG. 6 is a block diagram illustrating a hardware configuration of a mobile terminal used as an information communication terminal in the system illustrated in FIG. 1;

FIG. 7 is a block diagram illustrating a software configuration of the mobile terminal used as an information communication terminal in the system illustrated in FIG. 1;

FIG. 8 is a block diagram illustrating a hardware configuration of a sensing data management server used as a data processing device in the system illustrated in FIG. 1;

FIG. 9 is a block diagram illustrating a software configuration of the sensing data management server used as a data processing device in the system illustrated in FIG. 1;

FIG. 10 is a flowchart illustrating a processing procedure and processing details of an operation executed by the sensing device illustrated in FIG. 3;

FIG. 11 is a flowchart illustrating a processing procedure and processing details of an operation executed by the mobile terminal illustrated in FIG. 7;

FIG. 12 is a flowchart illustrating a processing procedure and processing details of an operation executed by the sensing data management server illustrated in FIG. 9;

FIG. 13 is a diagram illustrating an appearance of a sensing device according to another embodiment of the invention; and,

FIG. 14 is a diagram illustrating an appearance of a sensing device according to yet another embodiment of the invention.

### DESCRIPTION OF EMBODIMENTS

Embodiment of this invention will be described below with reference to the drawings.

### First Embodiment; Configuration Example; (1) System

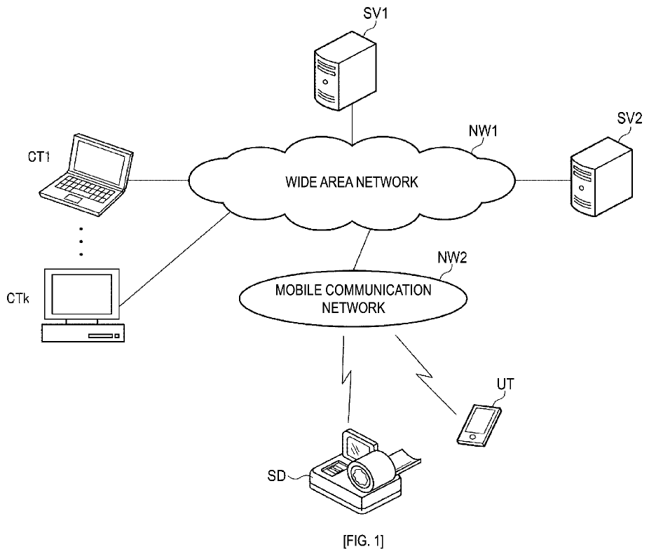

FIG. 1 is a diagram illustrating an example of an overall configuration of a sensing data management system according to a first embodiment of the invention.

In FIG. 1, for example, a sensing device SD is fixedly installed in a facility to which an unspecified number of users visits, such as a medical institution, a fitness club, a conference hall, and a commercial facility. Meanwhile, a sensing data management server SV1 functioning as a data processing device is installed on, for example, a cloud or a web.

The system according to the first embodiment makes it possible to transmit sensing data measured by the sensing device SD to the sensing data management server SV1 via a mobile communication network NW2 and a wide area network NW1.

The mobile communication network is not limited to one mobile communication network operated by one communication service provider, and a plurality of mobile communication networks each operated by a plurality of communication service providers may be mixed. In addition, for example, although Long Time Evolution (LTE (trade name)) is assumed as a radio access scheme of the mobile communication network, the radio access scheme is not limited thereto and may adopt 4G or 5G.

The wide area network NW1 includes the Internet and an access network used for accessing the Internet. Examples of the access network include, but are not limited to, a public data line provided by the communication service provider, a local area network (LAN), a wireless LAN, and a cable television (CATV) line.

In addition, the system according to the first embodiment enables data transmission between the sensing data management server SV1, a map information management server SV2, and user terminals CT1 to CTk via the wide area network NW1.

The map information management server SV2 stores and manages map information of an area including at least a target area in which the sensing device SD is installed in a database and discloses the map information free of charge.

The user terminals CT1 to CTk are used by, for example, the subject himself/herself or his/her family members, and users of the sensing data such as medical institutions, pharmaceutical manufacturers, and insurance companies to access the sensing data management server SV1 and view the sensing data of the subject.

Furthermore, the system according to the first embodiment makes it possible to notify the sensing device SD of the identification information unique to the subject (hereinafter referred to as a subject ID) via the mobile communication network NW2, for example, by using an information communication device carried by the subject, for example, a mobile terminal UT such as a smartphone.

(2) Device

(2-1) Sensing Device SD

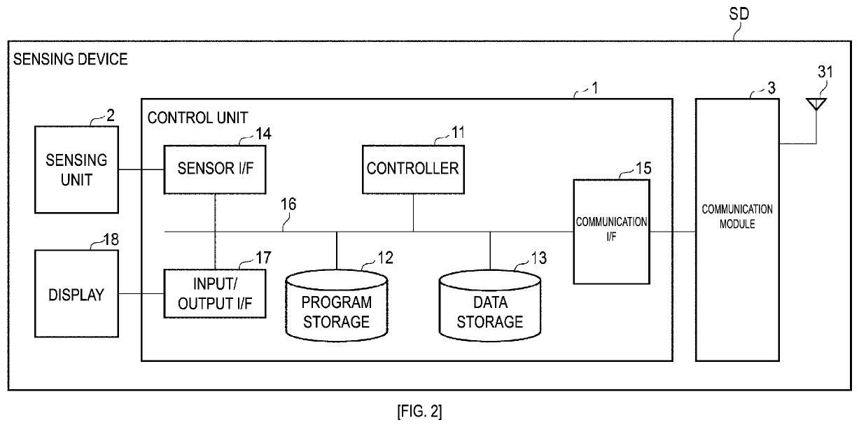

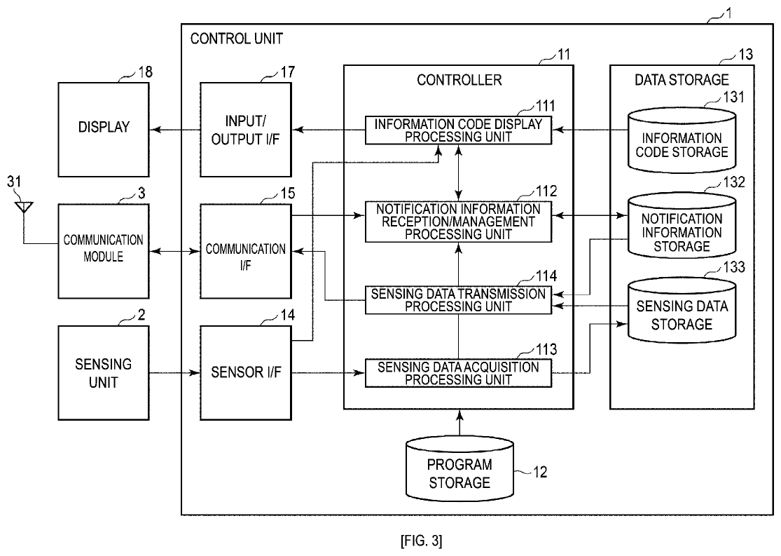

FIG. 2 and FIG. 3 are block diagrams respectively illustrating a hardware configuration and a software configuration of the sensing device SD.

The sensing device SD is constituted by a blood pressure monitor, for example, and includes a control unit 1, a sensing unit 2, and a communication module 3. The sensing unit 2 performs an operation of measuring sensing data such as blood pressure data under control of the control unit 1 and outputs the measured sensing data to the control unit 1.

The sensing unit 2 may be a device that measures, in addition to blood pressure data, other pieces vital data such as heart rates, electrocardiographic waveforms, blood sugar levels, amounts of activity, and stress level alone or in combination.

The communication module 3 is used to establish a wireless link with the mobile communication network NW2 in accordance with, for example, the LTE standard and transmit sensing data using the established wireless link. In addition, the communication module 3 is also used to receive emails, messages, or SNS transmission data transmitted from the mobile terminal UT.

The communication module 3 is not limited to a unit incorporated in the sensing device SD and may be a unit externally provided as an attached communication adapter or may be a communication module provided in an IoT device, a router, or a set-top box (STB) which is independently used.

The control unit 1 includes a controller 11 that uses a hardware processor such as a central processing unit (CPU). In addition, a storage unit including a program storage 12 and a data storage 13, a sensor interface (“interface” will be denoted by “I/F” below) 14, a communication I/F 15, and an input/output I/F 17 are connected to the controller 11 via a bus 16.

The sensor I/F 14 transmits and receives various control signals related to operations of the sensing unit 2 and receives sensing data output from the sensing unit 2 under control of the controller 11. The communication I/F 15 exchanges information data and the like with the communication module 3 under control of the controller 11. A display 18 and input/output devices such as operation buttons and a printer are connected to the input/output I/F 17.

The program storage 12 is configured by combining, for example, a non-volatile memory on which writing and reading can be performed as needed such as a hard disk drive (HDD) or a solid state drive (SSD) and a non-volatile memory such as a read only memory (ROM) as a storage medium and stores programs used for executing various kinds of control processing according to the first embodiment, in addition to middleware such as an operating system (OS).

The data storage 13 is a combination of, for example, a non-volatile memory on which writing and reading can be performed as needed such as an HDD or an SSD, and a volatile memory such as a random access memory (RAM) as a storage medium and includes an information code storage 131, a notification information storage 132, and a sensing data storage 133 as main storages used for implementing the first embodiment.

The information code storage 131 stores, for example, an information code obtained by encoding a communication address assigned to the sensing device SD in advance by a communication service provider of the mobile communication network NW2. As the communication address, for example, a telephone number or an email address of the mobile communication network NW2 is used.

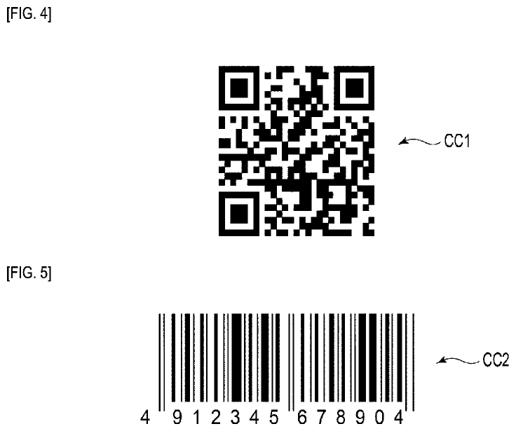

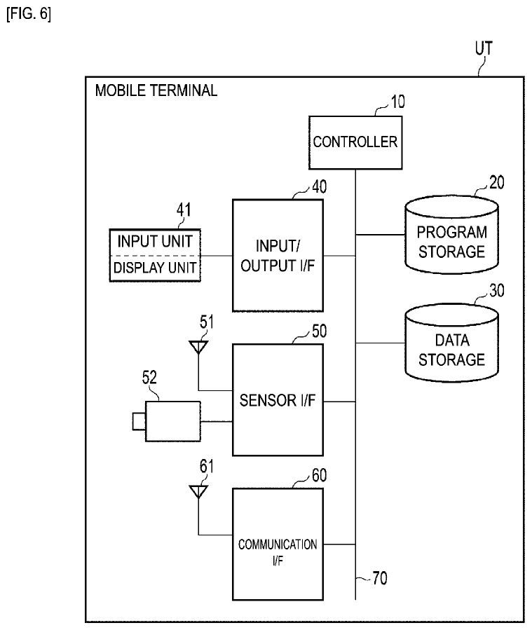

FIG. 4 and FIG. 5 illustrate examples of a QR code CC1 and a one-dimensional bar code each used as the information code. In addition to an optically readable code such as a QR code or a bar code, a code such as a wireless tag that outputs a code by radio or infrared rays may be used as the information code.

The notification information storage 132 is used to store notification information transmitted from the mobile terminal UT of the subject.

The sensing data storage 133 is used to store sensing data measured by the sensing unit 2.

The controller 11 includes an information code display processing unit 111, a notification information reception/management processing unit 112, a sensing data acquisition processing unit 113, and a sensing data transmission processing unit 114 as processing functions used for implementing the first embodiment. All of these processing units 111 to 114 are implemented by causing the hardware processor of the controller 11 to execute a program stored in the program storage 12.

The information code display processing unit 111 reads the information code from the information code storage 131 and outputs the information code from the input/output I/F 17 to the display 18 for display when the sensing unit 2 detects a behavior of the subject which is a sign of a sensing operation. As the behavior of the subject which is a sign of a sensing operation, for example, a situation in which the subject is sitting on in front of the sensing device SD or a motion of the subject inserting his/her arm into a cuff has been detected is assumed.

Subsequently, the information code display processing unit 111 monitors whether the information code display time has passed a first time set in advance while monitoring reception of the notification information from the mobile terminal UT during the information code display period. Then, when the notification information has been received or the first time elapsed without receiving the notification information, the information code display processing unit 111 performs processing to terminate the display of the information code.

The notification information reception/management processing unit 112 includes the following processing functions.

(1) Processing of receiving notification information transmitted from the mobile terminal UT via the communication module 3 and storing the received notification information in the notification information storage 132.

(2) Processing of monitoring whether a sensing operation has been started within a second time set in advance after the notification information is stored and deleting the notification information stored in the notification information storage 132 when the second time has elapsed without starting a sensing operation.

(3) Processing of monitoring whether a sensing operation has been performed again within a third time set in advance after execution of the sensing operation ends and deleting the notification information stored in the notification information storage 132 when the third time has elapsed without performing a sensing operation again.

The sensing data acquisition processing unit 113 performs processing of receiving the sensing data measured by the sensing unit 2 via the sensor I/F 14 and causing the sensing data storage 133 to store the received sensing data.

The sensing data transmission processing unit 114 reads sensing data that has not been transmitted from the sensing data storage 133 each time new sensing data is acquired by the sensing data acquisition processing unit 113, each time pieces of sensing data corresponding to a predetermined number of measurements are accumulated in the sensing data storage 133, or each time a predetermined time elapses from the previous transmission. Then, the sensing data transmission processing unit 114 performs processing of transmitting, from the communication module 3 to the sensing data management server SV1, the read sensing data together with the subject ID stored in the notification information storage 132 and the device ID, which is the individual identification information of the sensing device SD that is stored in advance in the sensing device SD.

2-2 Mobile Terminal UT

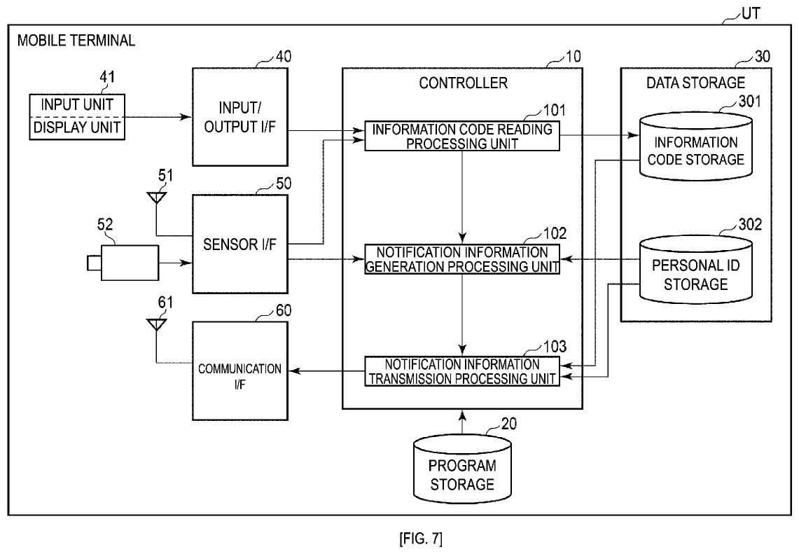

FIG. 6 and FIG. 7 are block diagrams respectively illustrating a hardware configuration and a software configuration of the mobile terminal UT.

The mobile terminal UT includes a general-purpose smartphone including, for example, a GPS position sensor, a camera, and a browser. As the mobile terminal UT, a tablet terminal, a wearable terminal, a laptop personal computer, or the like may be used as long as the terminal has the same functions.

The mobile terminal UT includes a controller 10 using a hardware processor such as a central processing unit. In addition, a storage unit including a program storage 20 and a data storage 30, an input/output I/F 40, a sensor I/F 50, and a communication I/F 60 are connected to the controller 10 via a bus 70.

An input/output device 41 is connected to the input/output I/F 40. The input/output device 41 is a device in which an input unit using a pressure-sensitive or a capacitive touch input sheet is arranged on a display screen of a display device using, for example, liquid crystal or organic EL and is used for inputting various kinds of operation data and displaying display data.

The sensor I/F 50 has a function of receiving GPS signals via an antenna 51 and a function of capturing image or video data captured by a camera 52. The camera 52 is used to optically read an information code including a QR code or a one-dimensional bar code in the present embodiment.

The communication I/F 60 performs information data transmission and reception with the sensing data management server SV1 by using a wireless access method and a communication protocol defined by the mobile communication network NW2, under control of the controller 10. In this example, LTE is used as the wireless access method.

The program storage 20 is configured by combining, for example, a non-volatile memory on which writing and reading can be performed as needed such as a HDD or an SSD and a non-volatile memory such as a ROM as a storage medium and stores programs used for executing various kinds of control processing according to the first embodiment, in addition to middleware such as an OS.

The data storage 30, for example is a combination of, includes, a non-volatile memory on which writing and reading can be performed as needed such as an SSD and a volatile memory such as a RAM as a storage medium and includes an information code storage 301 and a personal ID storage 302 as main storages used for implementing the first embodiment.

The information code storage 301 is used to temporarily store the information code read by the camera 52. The personal ID storage 302 is used to store unique identification information (hereinafter referred to as a “personal ID”) of the owner of the mobile terminal. In this example, because the subject of the sensing data uses his/her own mobile terminal to read the information code, the personal ID stored in the personal ID storage 302 represents the unique identification information of the subject.

As processing functions according to the first embodiment, the controller 10 includes an information code reading processing unit 101, a notification information generation processing unit 102, and a notification information transmission processing unit 103. All of these processing units 101 to 103 are implemented by causing the hardware processor of the controller 10 to execute programs stored in the program storage 20.

The information code reading processing unit 101 activates the camera 52 in response to a code reading operation in the input/output device 41 and reads the image data of the information code captured by the camera 52 via the sensor I/F 50. Then, the information code reading processing unit 101 recognizes the information code from the read image data and performs processing of storing the recognized information code in the information code storage 301.

When the information code reading processing unit 101 performs the information code reading process, the notification information generation processing unit 102 reads the personal ID of the owner of the mobile terminal UT, that is, the subject, from the personal ID storage 302 and performs processing of generating notification information including the read personal ID. The notification information generation processing unit 102 may acquire the position information of the mobile terminal UT measured by the GPS position measurement function from the sensor I/F 50 and add the acquired position information to the notification information.

The notification information transmission processing unit 103 performs processing of transmitting the generated notification information from the communication I/F 60 to the sensing device SD based on the address information of the sensing device SD included in the information code.

(2-3) Sensing Data Management Server SV1

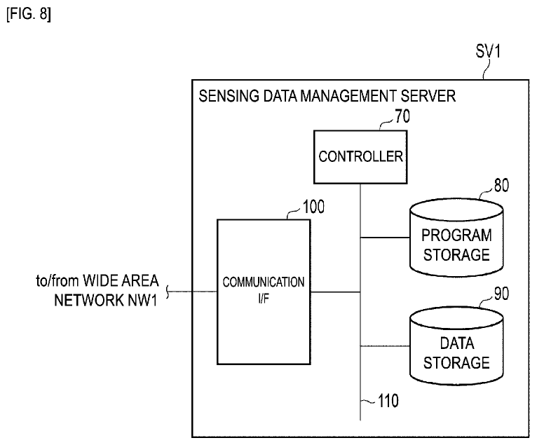

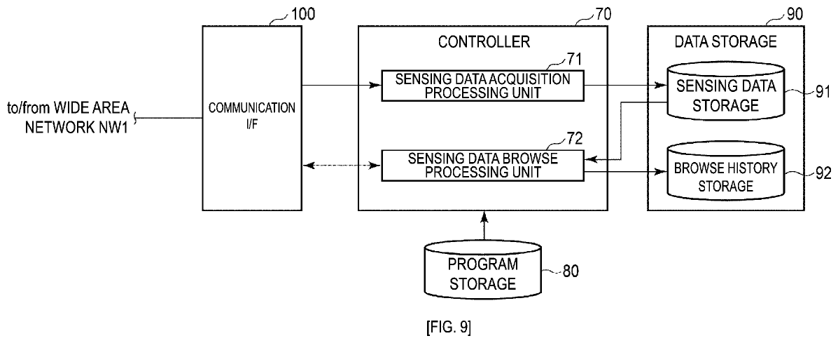

FIG. 8 and FIG. 9 are block diagrams respectively illustrating a hardware configuration and a software configuration of the sensing data management server SV1.

The sensing data management server SV1 is configured by, for example, a server computer, and includes a controller 70 using a hardware processor such as a central processing unit. In addition, a storage unit including a program storage 80 and a data storage 90 and a communication I/F 100 are connected to the controller 70 via a bus 110.

The communication I/F 100 transmits and receives information data to and from the sensing device SD, the mobile terminal UT, the map information management server SV2, and the user terminals CT1 to CTk by using a communication protocol defined by the wide area network NW1 under control of the controller 70.

The program storage 80 is configured by combining, for example, a non-volatile memory on which writing and reading can be performed as needed such as a HDD or an SSD and a non-volatile memory such as a ROM as a storage medium and stores programs used for executing various kinds of control processing according to the first embodiment, in addition to middleware such as an OS.

The data storage 90 is a combination of, for example, a non-volatile memory on which writing and reading can be performed as needed such as a HDD or an SSD and a volatile memory such as a RAM as a storage medium and includes a sensing data storage 91 and a browse history storage 92 as main storages used for implementing the first embodiment.

The sensing data storage 91 is used to store the sensing data transmitted from the sensing device SD in association with the device ID of the sensing device SD and the personal ID of the subject transmitted together with the sensing data.

The browse history storage 92 is used to store browse history information generated by the controller 70 when the sensing data is transmitted to the requesting user terminals CT1 to CTk in response to a browse request from the user terminals CT1 to CTk.

The controller 70 includes a sensing data acquisition processing unit 71 and a sensing data browse processing unit 72 as processing functions according to the first embodiment. All of these processing units 71 and 72 are implemented by causing the hardware processor of the controller 70 to execute programs stored in the program storage 80.

The sensing data acquisition processing unit 71 performs processing of receiving sensing data via the communication I/F 100 each time the sensing data is transmitted from the sensing device SD and causing the sensing data storage 91 to store the received sensing data in association with the device ID of the sensing device SD that is the transmission source and the personal ID of the subject.

When a browse request is sent from the user terminals CT1 to CTk, the sensing data browse processing unit 72 reads the sensing data associated with the personal ID designated by the browse request from the sensing data storage 91. Then, the sensing data browse processing unit 72 performs processing of transmitting the read sensing data from the communication I/F 100 to the requesting user terminals CT1 to CTk.

### Operation Example

Next, an example of an operation of the sensing data management system configured as described above will be described. In this example, description will be provided on the assumption that information code obtained by encoding the address information of the sensing device SD is stored in advance in the information code storage 131 of the sensing device SD.

(1) Input of Personal ID of Subject

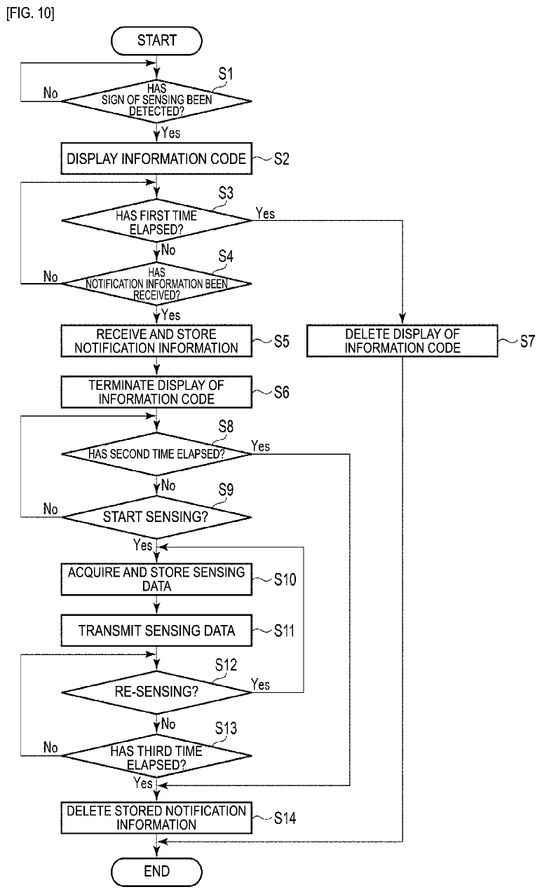

FIG. 10 is a flowchart illustrating a processing procedure and processing details of an operation executed by the controller 11 of the sensing device SD.

In a standby state, the controller 11 of the sensing device SD monitors, for example, a behavior that will be a sign of a sensing operation by the subject as a behavior related to a sensing operation by the subject. It is assumed that, in that state, the subject is seated at a position in front of the sensing device SD or performs a motion of inserting his/her arm into a cuff. In this case, the sensing device SD detects the behavior that will be a sign by, for example, a proximity sensor included in the sensing unit 2 or a sensor provided in the cuff.

When the controller 11 of the sensing device SD detects the behavior that will be a sign in step S1, the controller 11 reads the information code from the information code storage 131, outputs the read information code from the input/output I/F 17 to the display 18 for display under control of the information code display processing unit 111 in step S2. Accordingly, for example, the QR code or the one-dimensional bar code as illustrated in FIG. 4 or FIG. 5, respectively, is displayed on the display 18 of the sensing device SD.

At this time, a guidance message “please read the information code with your mobile terminal” may be displayed together on the display 18. A similar voice message may also be output from a speaker, which is not illustrated.

In this state, it is assumed that the subject activates a code reading application on his/her own mobile terminal UT and performs an operation of reading the information code displayed on the display 18. Then, the controller 10 of the mobile terminal UT executes a series of processing operations from the processing of reading the information code to the processing of transmitting the notification information as follows.

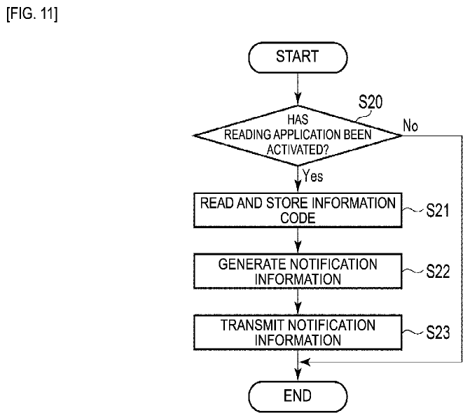

FIG. 11 is a flowchart illustrating a processing procedure and processing details executed by the controller 10 of the mobile terminal UT.

That is, when the controller 10 of the mobile terminal UT first detects the operation of activating the code reading application in step S20, the controller 10 activates the camera 52 in step S21 under control of the information code reading processing unit 101 and reads the image data of the information code captured by the camera 52 from the sensor I/F 50. Then, the controller 10 performs processing of recognizing the information code from the read image data and causing the information code storage 301 to store the recognized information code.

When the processing of reading the information code is completed, the controller 10 of the mobile terminal UT then reads the information code from the information code storage 301 in step S22 under control of the notification information generation processing unit 102. Then, the controller 10 reads the personal ID of the subject from the personal ID storage 302 and generates notification information including the read personal ID. Subsequently, the controller 10 transmits the notification information from the communication I/F 60 to the sensing device SD by using, for example, an email, a message, or an SNS message based on the address information of the sensing device SD included in the read information code, for example, a telephone number or an email address, under control of the notification information transmission processing unit 103 in step S23.

On the other hand, in a state in which the information code is displayed, the controller 11 of the sensing device SD monitors reception of the notification information in step S4 while monitoring whether a first time has elapsed since the start of display of the information code in step S3. In this state, if the notification information transmitted from the mobile terminal UT is received within the first time, the controller 11 receives the notification information and stores the received notification information in the notification information storage 132 in step S5 under control of the notification information reception/management processing unit 112.

When the storage of the notification information is completed, the notification information reception/management processing unit 112 notifies the information code display processing unit 111 of the completion. Upon receiving this notification, the information code display processing unit 111 terminates the display of the information code on the display 18 in step S6. If the notification information is not received within the first time, the controller 11 determines that the subject has not performed the processing of reading the information code, ends, by using the information code display processing unit 111, the display of the information code on the display 18 in step S7, and returns to the standby state as it is.

Thus, in this case, the sensing operation cannot be performed in the sensing device SD, and the trouble that only the sensing data is transmitted to the sensing data management server SV1 without input of the personal ID of the subject is suppressed from occurring. In this case, the sensing device SD causes the display 18 to display, for example, a guidance message such as “please perform an operation for sensing from the beginning” or causes a speaker to output a voice message with similar content.

(2) Sensing Operation

When the notification information is received and stored, the controller 11 of the sensing device SD monitors whether the sensing operation has been started in step S9 while monitoring whether a second time has elapsed since the storage of the notification information in step S8 under control of the sensing data acquisition processing unit 113.

In this state, for example, when the subject presses the sensing start button, the cuff contracts in the sensing unit 2, thereby a sensing operation for a blood pressure value is performed. In step S10, the sensing data acquisition processing unit 113 acquires the sensing data measured by the sensing unit 2 from the sensor I/F 14 and temporarily stores the acquired sensing data in the sensing data storage 133.

Subsequently, the controller 11 of the sensing device SD reads the sensing data from the sensing data storage 133 and reads the personal ID of the subject from the notification information storage 132 in step S11 under control of the sensing data transmission processing unit 114. Then, the controller 11 outputs the sensing data from the communication I/F 15 to the communication module 3 in a state of being associated with the personal ID of the subject and the device ID of the sensing device SD and transmits the sensing data from the communication module 3 to the sensing data management server SV1.

If the second time elapses without performing the sensing operation, the notification information reception/management processing unit 112 determines that the subject has given up the sensing operation and proceeds to step S14 to delete the notification information stored in the notification information storage 132. Thus, it is possible to suppress the trouble that the notification information of the subject who has given up the sensing operation from remaining in the notification information storage 132 as it is.

In addition, the sensing data acquisition processing unit 113 monitors whether the sensing operation has been performed again in step S12 while monitoring whether a third time has elapsed in step S13, after the transmission of the sensing data. When the subject performs a re-sensing operation in this state, the sensing operation is performed again in the sensing unit 2.

In this case, the sensing data acquisition processing unit 113 acquires the sensing data measured in the re-sensing operation from the sensor I/F 14 and causes the sensing data storage 133 to store the acquired sensing data in step S10. Then, the sensing data acquired in the re-sensing is transmitted from the communication module 3 to the sensing data management server SV1 together with the personal ID of the subject and the device ID of the sensing device SD stored in the notification information storage 132 in step S11 under control of the sensing data transmission processing unit 114.

In general, when blood pressure is measured, measurement values vary in most measurements, and thus in such a case, the subject may try measurement again. Thus, when a subject attempts to perform a sensing operation again within the third time after the first sensing operation ends as described above, the subject does not need to perform the operation of reading the information code again when he or she performs re-sensing by enabling the sensing operation to be continuously performed again.

If the third time elapses without performing the re-sensing operation, the notification information reception/management processing unit 112 transitions from step S13 to step S14 and deletes the notification information of the subject stored in the notification information storage 132. Then, the sensing device SD returns to the standby state. Thus, it is possible to suppress the trouble that the notification information of the subject from remaining in the notification information storage 132 as it is after the sensing operation ends. For this reason, a trouble that, even if another person subsequently performs a sensing operation, the sensing data is transmitted in association with the personal ID of the subject who was subjected to the sensing operation immediately before is suppressed.

In the above description, the sensing data is transmitted to the sensing data management server SV1 each time sensing data is acquired. However, sensing data that has not been transmitted may be caused to be read from the sensing data storage 133 and collectively transmitted each time a certain number of pieces of sensing data are accumulated in the sensing data storage 133 or each time a certain period of time elapses.

(3) Data Management of Sensing Data Management Server SV1

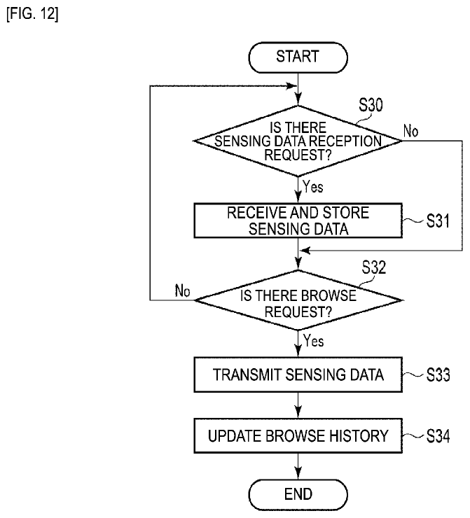

When the sensing device SD transmits sensing data, the controller 70 of the sensing data management server SV1 executes the following processing. FIG. 12 is a flowchart illustrating a processing procedure and processing details of the controller 70.

The controller 70 of the sensing data management server SV1 monitors the reception request sent from the sensing device SD in step S30. When a sensing data reception request is detected in step S30 in this state, in step S31, the controller 70 receives the sensing data transmitted from the sensing device SD via the communication I/F 100 and causes the sensing data storage 91 to store the received sensing data in association with the personal ID of the subject and the device ID of the sensing device SD that is the transmission source, which are received together with the sensing data, under control of the sensing data acquisition processing unit 71.

It is assumed in this state that, for example, a browse request designating the personal ID of the subject is transmitted from one of the user terminals CT1 to CTk used by the subject, his/her family member, or a family doctor. In this case, when the browse request is received in step S32, in step S33, the controller 70 of the sensing data management server SV1 reads, from the sensing data storage 91, the sensing data corresponding to the personal ID designated by the browse request under control of the sensing data browse processing unit 72 and transmits the read sensing data from the communication I/F 100 to any of the user terminals CT1 to CTk that is the request source. Then, after the transmission processing, the controller 70 generates browse information including, for example, the transmission destination and the transmission date and time of the sensing data and the personal ID of the subject as the browse target and causes the browse history storage 92 to store the generated browse information.

The sensing data management server SV1 may generate statistical information representing the mean of the plurality of pieces of sensing data, a variation tendency of blood pressure values for each personal ID for the plurality pieces of sensing data stored in the sensing data storage 91, store the generated statistical information also in the sensing data storage 91, and allow the statistical information to be browsed.

Actions and Effects

According to the first embodiment described above, the following actions and effects are exhibited. (1) When the sensing device SD detects a behavior that is a sign of a sensing operation by a subject, an information code obtained by encoding address information of the sensing device SD is read from the information code storage 131 and displayed on the display 18. Then, when the subject reads the information code with his/her own mobile terminal UT in that state, the mobile terminal UT generates notification information including the personal ID of the owner of the mobile terminal UT as the personal ID of the subject and transmits the generated notification information to the sensing device SD in accordance with the address information included in the information code. Meanwhile, upon receiving the notification information, the sensing device SD transmits the sensing data of the subject acquired from the sensing operation within the second time after the reception of the notification information to the sensing data management server SV1 together with the personal ID of the subject included in the notification information.

Thus, the sensing data of the subject together with his/her personal ID can be transmitted from the sensing device SD to the sensing data management server SV1. For this reason, the sensing device SD does not need to be provided with an additional communication module for near-field wireless data communication to transmit the sensing data to the mobile terminal UT, and thereby product costs of the sensing device SD can be kept low.

In addition, because the subject may only perform an operation of reading the information code by using the code reading function of the mobile terminal UT, the subject does not need to manually input his/her personal ID by operating the sensing device, and thereby the operation burden on the subject can be reduced and incorrect input of the personal ID can be suppressed.

Furthermore, because the information code is displayed on the display 18 of the sensing device SD, there is no need to print the information code on the housing of the sensing device SD or no need to produce and attach a seal or the like with the information code printed thereon.

(2) When the sensing device SD does not receive notification information within the first time after the information code is displayed, the sensing device SD automatically returns to the standby state after the display of the information code is deleted. For this reason, the trouble that a sensing operation is performed with no notification information including the personal ID of the subject received from the mobile terminal UT and the sensing data of the sensing operation is transmitted to the sensing data management server SV1 without identification can be suppressed.

(3) If no sensing operation is performed within the second time after the sensing device SD receives the notification information, the notification information is deleted from the notification information storage 132. For this reason, the trouble that unique identification information of the subject unnecessarily remains in the sensing device and thus the unique identification information is transmitted in association with sensing data of another subject can be suppressed.

(4) If a sensing operation is performed again within the third time after one piece of sensing data of the subject is transmitted, the sensing device SD allows the re-sensing and transmits the sensing data obtained thereby as it is as sensing data of the subject together with the personal ID. Thus, if one subject performs a sensing operation again within the third time, the sensing operation can be continuously performed without performing an operation of reading information code again, and thereby the operation burden on the subject can be reduced.

Variations

The case in which the notification information including only the personal ID of the subject is transmitted from the mobile terminal UT to the sensing device SD has been described in the first embodiment. However, the notification information may include position information measured by using the GPS position measurement function of the mobile terminal UT. In this case, the sensing device SD transmits the position information together with the personal ID included in the notification information when transmitting the sensing data of the subject to the sensing data management server SV1. In this way, the sensing data management server SV1 can estimate the name, the address, or the like of the facility indicating the sensing location of the subject based on, for example, the position information received together with the sensing data and acquired or stored map information.

In this way, the sensing data management server SV1 can manage the sensing data of the subject for each sensing location, and thereby statistics data representing a tendency of blood pressure of residents of each region, for example, can be generated and provided.

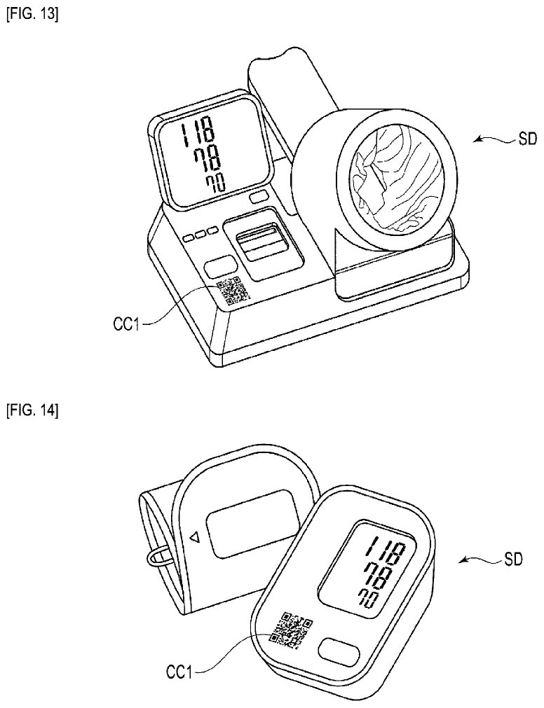

The case in which the information code is displayed on the display 18 of the sensing device SD has been described in the first embodiment. However, this invention is not limited thereto, and for example, information code CC1 may be attached or printed on a surface of the housing of the sensing device SD as illustrated in FIG. 13 or FIG. 14. In addition, in this case, the position at which the information code is attached or printed may be a front surface or a side surface of the housing, may be a top surface of a table on which the sensing device SD is installed, or may be a wall surface in front of or to the side of the table.

In addition, the information code may be of a type such as a wireless tag in which code information is transmitted by a wireless signal and is read by the mobile terminal UT when the mobile terminal UT receives the wireless signal, in addition to a code that is optically readable, such as a QR code CC1 and a one-dimensional bar code CC2. Furthermore, the unique identification information of the subject may include, in addition to the personal ID, personal attribute information such as the name and contact information of the subject.

Furthermore, the case in which the sensing data management server SV1 collects and manages the sensing data obtained by the one sensing device SD has been described as an example in the first embodiment. However, the sensing data management server SV1 may collect and manage pieces of sensing data respectively obtained by a plurality of sensing devices installed in the same facility or each in a plurality of other facilities, for example. In this case, the pieces of sensing data respectively obtained by a plurality of sensing devices installed in the same facility or a plurality of sensing devices respectively installed in a plurality of facilities in the same region may be integrated for each facility or each region to generate statistical information of the sensing data integrated for each facility or each region.

In addition, types, functions, processing procedures, processing details, and the like of the sensing device, the data processing device, and the information communication terminals can be variously modified without departing from the scope of the present invention.

### Second Embodiment

In the first embodiment, the mobile terminal UT directly accesses the sensing device SD based on the address information of the sensing device SD included in the information code. Thus, the address information of the sensing device SD, for example, the telephone number or the email address is opened to the public, and there is concern that the address information of the sensing device SD may leak to a third party, which may cause an unauthorized access.

In view of this, a second embodiment employs a method of accessing the sensing device SD from the mobile terminal UT via, for example, the sensing data management server SV1. In the second embodiment, the basic configuration of the system is the same as that in the first embodiment, and thus description will be made with reference to FIG. 1 to FIG. 9.

An information code includes, for example, the URL of the sensing data management server SV1 and information for individually identifying the sensing device SD (device ID). As the device ID, for example, a serial number of a product, or IMEI, IMSI, or SIM ICCID of a communication module mounted in the sensing device SD is used. The information code may be generated and displayed by the sensing device SD, or a seal or the like on which the information code is printed may be attached to the housing or the like of the sensing device SD.

When the controller 10 of the mobile terminal UT reads the information code under control of the information code reading processing unit 101, the notification information generation processing unit 102 generates first notification information including the device ID and the individual ID of the subject included in the information code. Then, under control of the notification information transmission processing unit 103, the controller 10 transmits the generated first notification information to the sensing data management server SV1 based on the URL included in the information code.

When the sensing data management server SV1 receives the first notification information transmitted from the mobile terminal UT via the communication I/F 100, the controller 70 reads address information of the sensing device SD registered in advance in association with the device ID included in the received first notification information from the database provided in the data storage 90. Then, the controller 70 transmits second notification information including the individual ID of the subject included in the first notification information from the communication I/F 100 to the sensing device SD by, for example, a message or an email based on the address information.

When the controller 11 of the sensing device SD receives the second notification information via the communication module 3 under control of the notification information reception/management processing unit 112, the controller 11 transmits the sensing data to the sensing data management server SV1 together with the individual ID of the subject included in the second notification information under control of the sensing data transmission processing unit 114.

On the other hand, upon receiving the second notification information under control of the sensing data acquisition processing unit 71, the controller 70 of the sensing data management server SV1 causes the sensing data storage 91 to store the sensing data and the individual ID of the subject, which are included in the second notification information, while maintaining the state in which the sensing data and the individual ID of the subject are associated with each other.

According to the second embodiment as described above, because the address information of the sensing device SD, for example, the telephone number and the email address thereof, is managed by the sensing data management server SV1, there is no concern that the address information of the sensing device SD may leak from the information code, and thus the problem of unauthorized access by others to the sensing device SD can be suppressed.

### Third Embodiment

According to a third embodiment of the present invention, the sensing data management server SV1 associates sensing data of a subject with the personal ID of the subject. Also in the third embodiment, the basic configuration of the system is the same as that in the first embodiment, and thus description will be made with reference to FIG. 1 to FIG. 9.

A device ID of the sensing device SD and the URL of the sensing data management server SV1 are included in an information code. Also in this example, for example, a serial number of a product, or IMEI, IMSI, or SIM ICCID of a communication module mounted in the sensing device SD is used as the device ID. The information code may be generated and displayed by the sensing device SD, or a seal or the like on which the information code is printed may be attached to the housing or the like of the sensing device SD.

When the controller 10 of the mobile terminal UT reads the information code under control of the information code reading processing unit 101, the notification information generation processing unit 102 generates notification information including the device ID of the sensing device SD included in the information code and the individual ID of the subject. Then, under control of the notification information transmission processing unit 103, the controller 10 transmits the generated notification information to the sensing data management server SV1 based on the URL included in the information code.

On the other hand, the sensing device SD transmits the measured sensing data of the subject to the sensing data management server SV1 together with the device ID of the sensing device SD under control of the sensing data transmission processing unit 114.

Upon receiving the sensing data and the notification information from the sensing device SD and the mobile terminal UT, respectively, via the communication I/F 100, the controller 70 of the sensing data management server SV1 first determines whether each reception timing is included in a predetermined period of time corresponding to, for example, a typical one measurement period. Then, if it is determined that the reception timing is included in the predetermined period of time, the controller 70 subsequently compares the device ID of the sensing device SD received together with the sensing data with the device ID of the sensing device SD included in the notification information. If the device IDs match, the controller 70 associates the sensing data and the individual ID of the subject that is included in the notification information with each other and causes the sensing data storage 91 to store the association result.

According to the third embodiment as described above, the sensing device SD does not need to perform the processing of receiving the notification information and processing of associating the sensing data of the subject with the individual ID, and thus the processing burden on the sensing device SD can be reduced. In addition, there is no need to assign address information to the sensing device SD, and thus unauthorized access to the sensing device SD can be suppressed.

### Fourth Embodiment

According to a fourth embodiment of the present invention, the mobile terminal UT associates sensing data of a subject measured by the sensing device SD with the individual ID of the subject and transmits the association result to the sensing data management server SV1. Also in the fourth embodiment, the basic configuration of the system is the same as that in the first embodiment, and thus description will be made with reference to FIG. 1 to FIG. 9.

Each time sensing data is measured, the controller 11 of the sensing device SD generates an information code including the sensing data and the URL of the sensing data management server SV1 under control of the information code display processing unit 111 and causes the display 18 to display the information code.

Upon reading the information code under control of the information code reading processing unit 101, the controller 10 of the mobile terminal UT associates the sensing data included in the information code with the individual ID stored in the mobile terminal UT and generates notification information under control of the notification information generation processing unit 102. Then, the controller 10 transmits the notification information to the sensing data management server SV1 based on the URL included in the information code under control of the notification information transmission processing unit 103.

Upon receiving the notification information via the communication I/F 100, the controller 70 of the sensing data management server SV1 causes the sensing data storage 91 to store the sensing data and the individual ID of the subject, which are included in the notification information, while maintaining the state in which the sensing data and the individual ID of the subject are associated with each other as they are under control of the sensing data acquisition processing unit 71.

According to the fourth embodiment as described above, the mobile terminal UT performs the processing of associating the sensing data of the subject measured by the sensing device SD with the individual ID of the subject. In addition, the sensing device SD is provided with a function of generating and displaying an information code including the sensing data, and there is no need to provide, in the sensing device SD, a wireless module such as Bluetooth, in order to transfer the sensing data to the mobile terminal UT. For this reason, it is possible to eliminate the need for any communication module from the sensing device SD, and thereby lower costs can be incurred for the sensing device SD.

### OTHER EMBODIMENTS

While embodiments of the present invention have been described in detail above, the foregoing description is merely illustrative of the present invention in all respects. It is a matter of course that various improvements and modifications can be made without departing from the scope of the present invention. That is, specific configurations in accordance with the embodiments may be adopted as appropriate at the time of carrying out the present invention.

In short, this invention is not limited to the embodiments as they are described above and can be embodied by modifying the components in an implementation stage in a range without departing from the gist thereof. In addition, various inventions can be formed by appropriately combining a plurality of components disclosed in the embodiments described above. For example, some components may be omitted from all the components described in the embodiments. Furthermore, the components of the different embodiments may be combined appropriately.

### REFERENCE NUMERALS LIST

SD Sensing device
SV1 Sensing data management server
SV2 Map information management server
CT1 to CTk User terminal
NW1 Wide area network
NW2 Mobile communication network
1 Control unit of sensing device
2 Sensing unit
3 Communication module
10, 11, 70 Controller
12, 20, 80 Program storage
13, 30, 90 Data storage
14, 50 Sensor I/F
15, 60, 100 Communication I/F
17, 40 Input/output I/F
18 Display
31, 51, 61 Antenna
41 Input/output device
52 Camera
71 Sensing data acquisition processing unit
72 Sensing data browse processing unit
91 Sensing data storage
92 Browse history storage
101 Information code reading processing unit
102 Notification information generation processing unit
103 Notification information transmission processing unit
111 Information code display processing unit
112 Notification information reception/management processing unit
113 Sensing data acquisition processing unit
114 Sensing data transmission processing unit
131 Information code storage
132 Notification information storage
133 Sensing data storage
301 Information code storage
302 Personal ID storage

1. A sensing data management system comprising:
a sensing device configured to measure sensing data of a subject;
a data processing device configured to perform data transmission with the sensing device via a network; and
an information communication terminal used by the subject and configured to perform communication with the sensing device via the network,
wherein the sensing data management system includes:
a code holder configured to hold, in a readable state, an information code obtained by encoding at least address information of the sensing device,
the information communication terminal includes:
a storage configured to store unique identification information of the subject;
a reading processing unit configured to read the information code from the code holder; and
a notification information transmission processing unit configured to generate notification information including at least the unique identification information of the subject stored in the storage and transmit the notification information to the sensing device based on the address information included in the information code, in response to a reading operation of the information code,
the sensing device includes:
a notification information reception processing unit configured to receive and store the notification information transmitted from the information communication terminal via the network; and
a sensing data transmission processing unit configured to transmit the sensing data of the subject to the data processing device together with the unique identification information included in the notification information, and
the data processing device includes:
a sensing data reception processing unit configured to receive the sensing data and the unique identification information transmitted from the sensing device via the network and hold the sensing data in association with the unique identification information.

2. The sensing data management system according to claim 1,
wherein the code holder is a printed medium where the information code is displayed in a state of being optically readable by the information communication terminal.

3. The sensing data management system according to claim 1,
wherein the code holder is a wireless tag that outputs the information code as a wireless signal readable by the information communication terminal.

4. An information communication terminal used in a system including a sensing device configured to measure sensing data of a subject, the data processing device configured to perform information communication with the sensing device via a network, the information communication terminal configured to perform information communication with the data processing device via the network, and a code holder configured to hold, in a readable state, an information code obtained by encoding at least address information of the sensing device, the information communication terminal comprising:
a storage configured to store unique identification information of the subject;
a reading processing unit configured to read the information code from the code holder; and
a notification information transmission processing unit configured to generate notification information including at least the unique identification information of the subject stored in the storage and transmit the notification information generated to the sensing device based on the address information included in the information code, in response to a reading operation of the information code.

5. The information communication terminal according to claim 4, further comprising
a position information measurement processing unit configured to measure position information corresponding to an installation location of the sensing device in conjunction with reading processing of the information code,
wherein the notification information transmission processing unit generates the notification information including the unique identification information stored in the storage and the position information and transmits the notification information to the sensing device based on the address information included in the information code.

6. A sensing device used in a system including the sensing device configured to measure sensing data of a subject, a data processing device configured to perform data transmission with the sensing device via a network, and an information communication terminal used by the subject and configured to perform communication with the sensing device via the network, the sensing device comprising:
an information code output processing unit configured to output an information code obtained by encoding at least address information of the sensing device in a state of being readable by the information communication terminal in association with a measurement operation of the sensing data;
a notification information reception processing unit configured to receive and store notification information transmitted, based on the information code, from the information communication terminal via the network, the notification information including at least unique identification information of the subject; and
a sensing data transmission processing unit configured to transmit the sensing data of the subject to the data processing device together with the unique identification information included in the notification information.

7. The sensing device according to claim 6,
wherein the information code output processing unit causes a display unit of the sensing device to display the information code in a state of being optically readable by the information communication terminal.

8. The sensing device according to claim 6,
wherein the information code output processing unit ends an output of the information code when a first time set in advance elapses after an output of the information code is started or when the notification information transmitted from the information communication terminal is received.

9. The sensing device according to claim 6,
wherein if measurement of the sensing data is not performed within a second time set in advance from a time point when the notification information is stored, the notification information reception processing unit deletes the notification information stored.

10. The sensing device according to claim 6,
wherein if re-measurement of the sensing data is not performed within a third time set in advance after the transmission of the sensing data is ended, the notification information reception processing unit deletes the notification information stored.

11. A sensing data transmission method executed by a system including a sensing device configured to measure sensing data of a subject, a data processing device configured to perform data transmission with the sensing device via a network, and an information communication terminal used by the subject and configured to perform communication with the sensing device via the network, the sensing data transmission method comprising:
reading, by the information communication terminal, an information code obtained by encoding at least address information of the sensing device;
by the information communication terminal, generating notification information including at least unique identification information of the subject stored in advance and transmitting the notification information to the sensing device based on the address information included in the information code, in response to a reading operation of the information code;
receiving and storing, by the sensing device, the notification information transmitted from the information communication terminal via the network; and
transmitting, by the sensing device, the sensing data of the subject to the data processing device together with the unique identification information included in the notification information.

12. A recording medium for causing a processor included in the information communication terminal according to claim 4 to perform processing of each of the processing units included in the information communication terminal.

13. A recording medium for causing a processor included in the sensing device according to claim 6 to perform processing of each of the processing units included in the sensing device.

14. A sensing data management system comprising:
a sensing device configured to measure sensing data of a subject;
a data processing device configured to perform data transmission with the sensing device via a network; and
an information communication terminal used by the subject and configured to perform communication with the sensing device via the network,
wherein the sensing data management system includes:
a code holder configured to hold, in a readable state, an information code obtained by encoding at least individual identification information of the sensing device and address information of the data processing device,
the information communication terminal includes:
a storage configured to store unique identification information of the subject;
a reading processing unit configured to read the information code from the code holder; and
a first notification information transmission processing unit configured to generate first notification information including at least the individual identification information of the sensing device included in the information code and the unique identification information of the subject stored in the storage and transmit the first notification information to the data processing device based on the address information included in the information code, in response to a reading operation of the information code,
the data processing device includes:
a processing unit configured to receive the first notification information transmitted from the information communication terminal via the network; and
a processing unit configured to acquire the address information of the sensing device associated in advance with the individual identification information of the sensing device included in the first notification information and transmit second notification information including at least the unique identification information of the subject included in the first notification information to the sensing device based on the address information of the sensing device, and
the sensing device includes:
a reception processing unit configured to receive and store the second notification information transmitted from the data processing device via the network; and
a sensing data transmission processing unit configured to transmit the sensing data of the subject to the data processing device together with the unique identification information of the subject included in the second notification information.

15. A sensing data management system comprising:
a sensing device configured to measure sensing data of a subject;
a data processing device configured to perform data transmission with the sensing device via a network; and
an information communication terminal used by the subject and configured to perform communication with the sensing device via the network,
wherein the sensing data management system includes:
a code holder configured to hold, in a readable state, an information code obtained by encoding at least individual identification information of the sensing device and address information of the data processing device,
the information communication terminal includes:
a storage configured to store unique identification information of the subject;
a reading processing unit configured to read the information code from the code holder; and
a notification information transmission processing unit configured to generate notification information including at least the individual identification information of the sensing device included in the information code and the unique identification information of the subject stored in the storage and transmit the notification information to the data processing device based on the address information included in the information code, in response to a reading operation of the information code,
the sensing device includes:
a sensing data transmission processing unit configured to transmit the sensing data of the subject to the data processing device together with the individual identification information of the sensing device, and
the data processing device includes:
a processing unit configured to receive the notification information transmitted from the information communication terminal via the network;
a sensing data reception processing unit configured to receive the sensing data transmitted from the sensing device and the individual identification information of the sensing device via the network; and
a sensing data reception processing unit configured to determine whether the individual identification information of the sensing device included in the notification information matches the individual identification information of the sensing device received together with the sensing data when the notification information and the sensing data are received within a predetermined period and associate the sensing data with the unique identification information of the subject included in the notification information if the individual identification information of the sensing device included in the notification information is determined to match the individual identification information of the sensing device received together with the sensing data.

16. A recording medium for causing a processor included in the information communication terminal according to claim 5 to perform processing of each of the processing units included in the information communication terminal.

17. A recording medium for causing a processor included in the sensing device according to claim 7 to perform processing of each of the processing units included in the sensing device.

18. A recording medium for causing a processor included in the sensing device according to claim 8 to perform processing of each of the processing units included in the sensing device.

19. A recording medium for causing a processor included in the sensing device according to claim 9 to perform processing of each of the processing units included in the sensing device.

20. A recording medium for causing a processor included in the sensing device according to claim 10 to perform processing of each of the processing units included in the sensing device.
